(19) United States
(12) Patent Application Publication
(10) Pub. No.: US 2018/0296466 A1
(43) Pub. Date: Oct. 18, 2018

(54) GELATIN AND COMPRESSION BINDER FOR USE IN ORAL ADHERING DISCS

(71) Applicant: OraHealth Corp., Seattle, WA (US)

(72) Inventor: Jeffrey T. Haley, Mercer Island, WA (US)

(73) Assignee: OraHealth Corp., Seattle, WA (US)

(21) Appl. No.: 15/901,707

(22) Filed: Feb. 21, 2018

Related U.S. Application Data
(60) Provisional application No. 62/485,964, filed on Apr. 16, 2017.

(51) Int. Cl. A61K 9/00; A61K 9/28; A23L 27/00; A23P 10/40; A23L 29/281

(52) U.S. Cl. CPC A61K 9/006; A61K 9/2873; A61K 9/2866; A61K 9/286; A23V 2002/00; A23L 27/00; A23P 10/40; A23L 29/284; A61K 9/284

(57) ABSTRACT

The disclosure provides a bi-layer, pressed particles, adhering troche, comprising, (a) a first layer of pressed particles comprising an ingredient to be released; (b) a second, adhesive layer of pressed particles comprising a mixture of at least 80% by weight gelatin particles and 1-15% by weight compression binder particles so that the gelatin particles have a greater tensile strength from one to another than if the gelatin particles had been pressed without the binder; wherein the troche has a first non-adhesive side and a second adhesive side; wherein the troche is at least 5 mm in two dimensions; and wherein the troche adhered in a mouth dissolves without smearing or breaking apart.

### CROSS-RELATED APPLICATIONS

This patent application claims priority from U.S. Provisional Patent Application No. 62/485,964, filed 16 Apr. 2017. This application incorporates U.S. A No. 62/485,964 in its entirety.

### TECHNICAL FIELD

This patent application relates generally to oral adhering discs or troches that contain gelatin and a compression binder in an adhesive layer.

### BACKGROUND

Oral adhering discs are used to time release ingredients into saliva in the mouth, into the mucosa to which the disc is adhered, and into mucosa that the disc touches while the disc is adhered to teeth or gums. A generic term that includes all such shapes is “adhering troche.” (Troche is a term for an object held in the mouth that releases ingredients to achieve an effect.) A common generic term is “oral adhering disc”, although as described herein, the adhering troches need not be disc shaped. They may be squarish or oval or oblong or any other shape that is roughly flat at least on one side.

Some oral adhering discs have two layers, a thin adhesive layer and a thicker, slowly dissolving layer that contains an ingredient for release. Common forms are made with a bi-layer tablet press. Many synthetic hydrophilic compounds are known for use as the adhesive layer, such as poly-acrylic acid, carbomer, carbopol, and povidone. An effective natural compound, in sufficient concentration, is acacia gum, also known as gum arabic. Unfortunately, some people are sensitive to acacia gum adhesive, or develop a sensitivity to it over weeks or months or years of daily use.

Another natural compound that is adhesive is gelatin. Gelatin may be liquefied with heat or with pressure (or both) or dissolved with water as is done for candy making and formed into a slab or disc. When cooled and dried, the gelatin disc adheres to oral mucosa. Unfortunately, gelatin particles in a suitable size range for making oral adhering discs by tablet compression are not sufficiently compressible with a tablet press to form a suitably non-friable a bi-layer adhering troche with a bi-layer tablet press.

### SUMMARY

The present disclosure provides a muco-adhesive composition that is effective for adhering troches and does not include acacia gum. The composition can be made by mixing or granulating gelatin particles with particles of a binder, e.g., cellulose or other gums, and pressing with a tablet press. In the commonly available granular forms of a suitable size, gelatin by itself is adequately adhesive, but it is not sufficiently compressible to form a strong, non-friable layer by pressing particles with a tablet press. A compression binder is required that does not interfere with long duration adhesion by the pressed particles of gelatin and does not swell to form a gelatinous blob with unattractive mouth feel.

In one aspect, an adhering troche is provided that has two sides, and when held in a human mouth, it adheres and remains in the mouth as a single item that does not smear or break apart, The troche may be made by a process comprising, (a) forming a first layer by compressing particles, wherein the first layer has a roughly flat side at least 5 mm in two dimensions and comprises an ingredient to be released into saliva and/or mucosa; (b) forming a second, adhesive layer by compressing particles, wherein the second, adhesive layer has a roughly flat side at least 5 mm in two dimensions and comprises at least 80% by weight gelatin particles mixed with a sufficient quantity of a suitable compression binder to bind the particles together when pressed in a tablet press; wherein the layers are compressed together, side to side, such that one side of the resulting troche is muco-adhesive and the other side of the resulting troche is not muco-adhesive.

In one aspect, a bi-layer, pressed particles, adhering troche, is manufactured. The troche comprises, (a) a first layer of pressed particles comprising an ingredient to be released; and (b) a second, adhesive layer of pressed particles comprising a mixture of at least 80% by weight gelatin particles and 1-15% by weight compression binder particles so that the gelatin particles have a greater tensile strength from one to another than if the gelatin particles had been pressed without the binder; wherein the troche has a first non-adhesive side and a second adhesive side; wherein the troche is at least 5 mm in two dimensions; and wherein the troche adhered in a mouth dissolves without smearing or breaking apart.

In embodiments, at least 80% of the particles of gelatin in the adhesive layer have a size in the range of 150 microns to 600 microns.

In some embodiments, the mixture of gelatin and compression binder are first mixed dry. In certain embodiments, the dry mixing further comprises dry granulation by compressing the particles together and then grinding to a suitable size. In other embodiments, the mixture of compression binder and gelatin are first mixed by a wet granulation process. In certain embodiments, the mixture formed by wet granulation is dried into granules. In yet other embodiments, the mixture comprises gelatin particles sprayed with compression binder that is suspended or dissolved in a liquid, whereupon the mixture is dried.

In embodiments, the adhesive layer adheres to a roof of a mouth of a human strongly enough to hold triple the weight of the troche against force of gravity. In certain embodiments, the adhesive layer adheres to a roof of a human mouth strongly enough to hold the weight of the troche plus an additional 680 mg against force of gravity.

In embodiments, the compression binder comprises 1% to 10% of the adhesive layer by weight. In other embodiments, the compression binder comprises cellulose gum at 2% to 8% of the adhesive layer by weight. In certain embodiments, the cellulose gum comprises hydroxy-propyl-cellulose or hydroxyl-propyl-methyl cellulose or carboxy-methyl-cellulose or croscarmellose. In some embodiments, at least 50% of the compression binder comprises a binder selected from the group consisting of gum karaya (tragacanth), locust bean gum, xanthan gum, pectin, konjac gum, tara gum, gellan gum, alginate, carrageenan, agar, starch, dextrin (malto-dextrin), hydrogenated starch hydrolysate, polyvinyl pyrrolidone, carbopol (poly-acrylic acid) and combinations of the binders.

These and other aspects of the present invention will become evident upon reference to the following detailed description and attached drawing.

### BRIEF DESCRIPTION OF THE DRAWING

FIG. 1 shows a cross section of a bi-layer adhering troche.

### DETAILED DESCRIPTION

The present disclosure provides an “adhering troche”, also called an “oral adhering disc” (terms used interchangeably herein), which is a device placed in a mouth or on a mucous membrane and dissolves (erodes) over time without disintegrating or smearing. The oral adhering disc is typically used to time release one or more ingredients into saliva in a mouth, into mucosa to which the disc is adhered, and into mucosa that the disc touches while the disc is adhered to teeth or gums.

The oral adhering discs of the present disclosure comprise two pressed layers: one layer comprises an adhesive and a compression binder, and a second layer comprises an ingredient to be released.

The adhesive layer comprises gelatin and a compression binder. Gelatin, which is derived from collagen, is an adhesive compound, and in particular, it is muco-adhesive. If gelatin particles are too small, they are difficult to work with for tablet pressing and do not adhere well to mucosa. If too large, they impart an unattractive, bumpy feel in the mouth. Within the context of the claimed invention, suitably sized particles of gelatin are in the range of 150-600 microns. Not all the particles need to be in this size range, but ideally at least 80% of the gelatin particles should be at least about 150 microns and less than about 600 microns.

The concentration of gelatin can be diluted somewhat and still be effective as an oral adhesive. The compression binder necessarily dilutes the gelatin. But if the gelatin is diluted too much it will lose its superior adhesive qualities. The concentration of gelatin in the adhesive layer is at least 80%. The optimal concentration of gelatin may be higher, such as at least 85%, at least 90%, or at least 95%. The optimal concentration may depend in part on the compression binder used. For example, when dextrin is the binder, and without other ingredients present, the adhesive layer may contain as little as 80% gelatin and as much as 20% dextrin. Other binders, such as hydroxy-propyl cellulose and carboxy-methyl cellulose, can be used in lower concentrations.

Gelatin is conveniently procured from commercial sources. The gelatin may be sourced from any animal, including fish. Both types A and B are suitable.

In common, commercially available dry granular forms, gelatin by itself is adequately adhesive. It is not, however, sufficiently compressible to form a strong layer when pressed in a tablet press. For at least this reason, a compression binder is added to the gelatin.

Any known compression binder, e.g. cellulose gum, that provides adequate binding without interfering with adhesion by the gelatin and allows the finished product to remain adequately shelf stable may be used. The exception is acacia gum because an objective is to avoid use of acacia gum. For commercialization, preferably, the binder is a molecule that occurs in nature and can, therefore, be labeled a “natural” binder. The natural binder may be gum karaya (tragacanth), locust bean gum, xanthan gum, pectin, konjac gum, tara gum, gellan gum, alginate, carrageenan, agar, starch, dextrin (malto-dextrin), hydrogenated starch hydrolysate, or cellulose gum (carboxy-methyl cellulose or caramellose or croscarmellose or hydroxy-propyl cellulose or hydroxy-propyl-methyl cellulose). Other suitable binders include a synthetic (non-natural) gum such as carbopol (polyacrylic acid) or polyvinyl pyrrolidone. The binder should not be acacia gum because some individuals have sensitivity to acacia gum. One or more of the binders may be used in a troche. For example, the troche may comprise gelatin and a single binder or gelatin and a blend of two or more binders.

While all of these binders are somewhat adherent to mucosa, they have disadvantages that make them unsuitable to be a major ingredient for the adhesive. All of the natural gums listed above have been tested and found to be unsuitable for a major ingredient for the adhesive for oral adhering discs, except for acacia gum. These disadvantages include: insufficiently adherent to mucosa and unattractive properties such as bad flavor or excessively acidic or swelling too much or turning to a gel that dissipates. Consequently the amount of binder is best minimized to in some embodiments a range of 1% to 15% by weight, in other embodiments 2% to 8%, and in still other embodiments 3%-6%. Overall, the compression binder is added in the least concentration to minimize adverse properties of the compression binder within a muco-adhesive layer, and to keep the gelatin concentration as high as possible to maintain maximum adhesive effectiveness.

Any compression binder that can be obtained in granular or powder form, will mix well with granular gelatin and stay mixed, and will remain shelf stable when mixed can be used. Two examples include carboxy-methyl-cellulose and hydroxy-propyl-cellulose. Effective ratios of hydroxy-propyl-cellulose to gelatin range from 1% to 15% compression binder by weight. A suitable formulation uses fine powder hydroxy-propyl-cellulose at a concentration of 2% to 8%, or from 3% to 6%. If the amount of hydroxy-propyl-cellulose is increased over about 8%, the adhesiveness of the gelatin is reduced.

The adhesive layer may comprise other ingredients in addition to gelatin and a compression binder. Other types of ingredients that can be used include an active ingredient to be released, a flavor, an anti-caking agent, or a press lubricant. The other ingredient or ingredients should be in a concentration range so as to not inhibit adhesiveness of the troche. For adequate adhesive strength, the gelatin should be at least 80%, and the compression binder should be in the range of 1% to 15%, with up to 19% other non-problematic additives, all by weight.

To be an effective adhesive layer for an oral adhering disc, the adhesive layer should adhere to a roof of a mouth of a human strongly enough to hold triple the weight of the disc against force of gravity. For example, if the disc (troche) weighs 750 mg, the adhesive should be strong enough to hold 2.25 g. To be an effective adhesive layer for an oral adhering disc weighing less than 340 mg, the adhesive layer should adhere to a roof of a mouth of a human strongly enough to hold the weight of the disc plus an additional 680 mg against force of gravity.

The non-adherent layer comprises one or more ingredients to be released into saliva or mucosa. The ingredient may be anything that is non-adherent to mucosa (or insignificantly adherent at concentration used). Active ingredients include pain relievers, flavorings, anti-bacterial compounds, medications, appetite suppressants, cough suppressants, expectorants, or any other desired ingredient. Other types of ingredients that can be used include a flavor, an anti-caking agent, or a press lubricant.

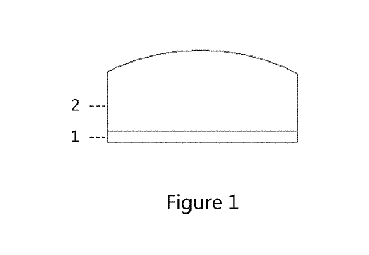

There are several suitable manufacturing methods. A preferred method is using a bi-layer tablet press. Either the adherent or non-adherent layer can be pressed first. The ingredients for each layer are typically dry and mixed together before being put into the tablet press. An exemplary method for making bi-layer troches using a typical press comprises placing the ingredient-releasing powder in the die sitting on the lower punch, tamping the powder with the upper punch, which leaves the surface having the shape of the upper punch face, adding powder of the adhesive layer, and then pressing with an upper punch. The shape of the upper punch that presses the ingredient-releasing powder and that presses the adhesive powder may be the same or different. Thus, a troche may have an adhesive side and an ingredient side with different shapes. For example, the ingredient-releasing powder may be placed on a rounded lower punch and tamped with a flat or essentially flat punch forming the shape shown in FIG. 1, and the adhesive powder tamped with the same flat or essentially flat punch. In another example, a troche has a dimpled adhesive face and a rounded ingredient face. An example of a bi-layer tablet is the adhering xylitol troche disclosed in U.S. patent application Ser. No. 11/800,381, filed May 4, 2007, which is incorporated in its entirety.

For ease of manufacturing, granular gelatin and a compression binder ingredient(s), such as powdered cellulose gum, are mixed together. The mixture is used directly in a tablet press. There are several ways to mix gelatin and a compression binder together. The methods can be generally categorized as dry mixing and wet mixing.

In one dry mixing method, dry particles of the binder are simply mixed with dry particles of the gelatin, and the resulting mixture pressed as one layer of a bi-layer troche with a bi-layer tablet press. In another dry mixing method, the particles binder may be adhered to particles of the gelatin by a dry granulation (compression) process, in which dry particles are mixed and then squashed until one of them deforms and is squashed against the other which holds them together. The resulting combined particles are broken (e.g., by grinding) to be small enough for pressing with a bi-layer tablet press. This process is sometimes called “dry granulation with pressure”. (see, Granulation_(process) in Wikipedia.)

The particles of the binder may be wet mixed with particles of the gelatin (wet granulation) and the resulting mixture then dried and broken into granules and the granules then pressed into a bi-layer troche with a bi-layer tablet press. The process of granulation ensures that the ingredients granulated together will have a constant ratio in any portion of the powder that is taken for any purpose, such as for pressing into tablets. In granulation, each resulting granule includes each of at least two ingredients adhered together within the granule so that they do not come apart from each other during subsequent processing.

The binder may be mixed with water or another solvent such as alcohol to form a dissolved mixture or a suspension or a slurry and sprayed onto particles of gelatin and the resulting mixture is then dried, sieved or ground as necessary, and pressed into a bi-layer troche with a bi-layer tablet press. In another wet mixing method, the binder may be mixed with water or another solvent such as alcohol to form a dissolved mixture and mixed with particles of gelatin to make a slurry and the resulting mixture then dried and ground or macerated to suitable particle size and then pressed into a bi-layer troche with a bi-layer tablet press.

The layers' ingredients may be pressed into a disc shaped tablet. The tablet punch that forms the adhesive side should be shaped for good contact with a surface in the mouth, usually essentially flat, sometimes with a bump in the center of the punch to create a dimple in the disc surface, sometimes cupped to give a domed surface such as for adhesion to the roof of the mouth. The other tablet press punch may be be cupped to provide a domed outer surface on a thick disc, or it may be nearly flat for a thin disc.

The troche may be any shape, such as round-ish, rectangular-ish, oval, or oblong. The precise shape is unimportant as long as it is not irritating in the mouth. The sides of the troche may be flat, approximately flat, convex or concave. In some embodiments, the troche is dimpled on one side.

The size of a troche will generally also take into account the ease and cost of manufacturing, surface intended for the troche to adhere, or consumer preference. In general, a troche will be at least 5 mm in at least two dimensions, or will range from about 5-20 mm, or from about 5-18 mm, or from 9-16 mm, or from about 7-18 mm, or from about 10-15 mm in at least two dimensions. When the troche is round or nearly round, the size range represents suitable diameters. The thickness of a troche will generally be from 1-10 mm, and more generally from 3-6 mm. The weight of a troche is typically 100-850 milligrams in weight.

The troches may be supplied as a kit with packaging and instructions. In general, a subject is instructed to place the troche against her teeth, gum or other oral mucosal surface. The troche adheres to the surface and dissolves over time. The troches are manufactured to dissolve over at least 30 minutes, often 1 hour, up to 4 hours when used while awake, up to 8 hours when used while sleeping. Depending on the purpose of the troche (e.g., stimulate salivation), the user maybe instructed to use a troche as often as needed, any time of day or night, only during the day or only at night. The active ingredient will be delivered into saliva or mucous membrane as the troche dissolves.

From the foregoing it will be appreciated that, although specific embodiments have been described herein for purposes of illustration, various modifications may be made without deviating from the spirit and scope of the invention. Accordingly, the invention is not limited except as by the appended claims.

I claim:

1. A bi-layer, pressed particles, adhering troche, comprising:
(a) a first layer of pressed particles comprising an ingredient to be released;
(b) a second, adhesive layer of pressed particles comprising a mixture of at least 80% by weight gelatin particles and 1-15% by weight compression binder particles so that the gelatin particles have a greater tensile strength from one to another than if the gelatin particles had been pressed without the binder;
wherein the troche has a first non-adhesive side and a second adhesive side;
wherein the troche is at least 5 mm in two dimensions; and
wherein the troche adhered in a mouth dissolves without smearing or breaking apart.

2. The adhering troche of claim 1, wherein at least 80% of the particles of gelatin in the adhesive layer have a size in the range of 150 microns to 600 microns.

3. The adhering troche of claim 1, wherein the mixture of gelatin and compression binder are first mixed dry.

4. The adhering troche of claim 3, wherein the dry mixing further comprises dry granulation.

5. The adhering troche of claim 1, wherein the mixture of compression binder and gelatin are first mixed by a wet granulation process.

6. The adhering troche of claim 5, wherein the mixture formed by wet granulation is dried into granules.

7. The adhering troche of claim 5, wherein the mixture comprises gelatin particles sprayed with compression binder that is suspended or dissolved in a liquid, wherein the mixture is dried.

8. The adhering troche of claim 1, wherein the adhesive layer adheres to a roof of a mouth of a human strongly enough to hold triple the weight of the troche against force of gravity.

9. The adhering troche of claim 1, wherein the adhesive layer adheres to a roof of a human mouth strongly enough to hold the weight of the troche plus an additional 680 mg against force of gravity.

10. The adhering troche of claim 1, wherein the compression binder comprises 1% to 10% of the adhesive layer by weight.

11. The adhering troche of claim 1, wherein the compression binder comprises cellulose gum at 2% to 8% of the adhesive layer by weight.

12. The adhering troche of claim 11, wherein the cellulose gum comprises one or more of hydroxy-propyl-cellulose or hydroxyl-propyl-methyl cellulose or carboxy-methyl-cellulose or croscarmellose.

13. The adhering troche of claim 11, wherein at least 50% of the compression binder comprises a binder selected from the group consisting of gum karaya (tragacanth), locust bean gum, xanthan gum, pectin, konjac gum, tara gum, gellan gum, alginate, carrageenan, agar, starch, dextrin (malto-dextrin), hydrogenated starch hydrolysate, polyvinyl pyrrolidone, carbopol (poly-acrylic acid) and combinations of the binders.
